SEQUENCE LISTING (72 sequences)

SEQ ID NO: 1 - DNA, 19 bp, Triticum aestivum
tgccggacacgtggcgcga

SEQ ID NO: 2 - DNA, 27 bp, Zea mays
ttcgagaagaaccgagacgtggcgggc

SEQ ID NO: 3 - DNA, 27 bp, Zea mays
gcgctcgcgccacgtgggcatgccgcc

SEQ ID NO: 4 - DNA, 25 bp, Zea mays
ggttgtcacatgtgtaaaggtgaag

SEQ ID NO: 5 - DNA, 28 bp, Zea mays
gatcatgcatgtcattccacgtagataa

SEQ ID NO: 6 - DNA, 20 bp, Cauliflower mosaic virus
gtggattgatgtgatatctc

SEQ ID NO: 7 - DNA, 28 bp, Cauliflower mosaic virus
tccactgacgtaagggatgacgcacaat

SEQ ID NO: 8 - DNA, 20 bp, Agrobacterium
tgacgtaagcgcttacgtca

SEQ ID NO: 9 - DNA, 24 bp, Nicotiana tabacum
gactaatggcggctcttatctcac

SEQ ID NO: 10 - DNA, 25 bp, Glycine max
gccctcgtgtctcctcaataagcta

SEQ ID NO: 11 - DNA, 27 bp, Glycine max
gcaatcctttgtctcaataagttccac

SEQ ID NO: 12 - DNA, 22 bp, Glycine max
aagggagacaacttgtctccca

SEQ ID NO: 13 - DNA, 24 bp, Pisum sativum
atcttgtgtggttaatatggctgc

SEQ ID NO: 14 - DNA, 25 bp, Arabidopsis thaliana
cttcatcttcttcctccaccaaacg

SEQ ID NO: 15 - DNA, 23 bp, Arabidopsis thaliana
atttcatggccgacctgcttttt

SEQ ID NO: 16 - DNA, 25 bp, Glycine max
agaagcttccagaagcttctagaag

SEQ ID NO: 17 - DNA, 20 bp, Zea mays
atgcacgaattgaccattcc

SEQ ID NO: 18 - DNA, 28 bp, Petroselinum crispum
cataagagccgccactaaaataagaccg

SEQ ID NO: 19 - DNA, 20 bp, Triticum aestivum
ggccacgtcaccaatccgcg

SEQ ID NO: 20 - DNA, 30 bp, Zea mays
cgggtcagtgtacctaccaaccttaaacac

SEQ ID NO: 21 - DNA, 28 bp, Zea mays
cgtctaactgcgactggcaggtgcgcac

SEQ ID NO: 22 - DNA, 29 bp, Petroselinum crispum
atccggtggccgtccctccaacctaacct

SEQ ID NO: 23 - DNA, 15 bp, Rice tungro bacilliform virus
ccagtgtgcccctgg

SEQ ID NO: 24 - DNA, 24 bp, Oryza sativa
taggttaattattggcggtaatta

SEQ ID NO: 25 - DNA, 26 bp, Artificial Sequence
  synthetic
aaacggtaaaaaagcggtagattacc

SEQ ID NO: 26 - DNA, 22 bp, Avena sativa
gaaatagcaaatgttaaaaata

SEQ ID NO: 27 - DNA, 27 bp, Glycine max
aaaaataatattaatattatattgaaa

SEQ ID NO: 28 - DNA, 30 bp, Arabidopsis thaliana
ataagctttaccattaatggtaaagcttgg

SEQ ID NO: 29 - DNA, 30 bp, Arabidopsis thaliana
caatactttccatttttagtaactaagctt

SEQ ID NO: 30 - DNA, 22 bp, Arabidopsis thaliana
ggtatcgttgaccgagttgact

SEQ ID NO: 31 - DNA, 26 bp, Petunia hybrida
ttgacagtgtcacttgacagtgtcac

SEQ ID NO: 32 - DNA, 18 bp, Zea mays
gatcaaaaaagtgagatc

SEQ ID NO: 33 - DNA, 31 bp, Petroselinum crispum
attcaatagtgtgctaattgtttaagagttg

SEQ ID NO: 34 - DNA, 22 bp, Hordeum vulgare
tgccattgccaccggcccccca

SEQ ID NO: 35 - DNA, 22 bp, Glycine max
agcagacatggtaggcagtgca

SEQ ID NO: 36 - DNA, 22 bp, Phaseolus vulgaris
tcacctaccctacttcctatcc

SEQ ID NO: 37 - DNA, 30 bp, Hordeum vulgare
aatcgtcatgaatgaagtcatgtgacggct

SEQ ID NO: 38 - DNA, 25 bp, Nicotiana tabacum
aggggcagcttcgacctccttctcc

SEQ ID NO: 39 - DNA, 31 bp, Artificial Sequence
  synthetic
tcagaacacgcaagttgccagctcacccaac

SEQ ID NO: 40 - DNA, 20 bp, Zea mays
agatatgcatgatctttaac

SEQ ID NO: 41 - DNA, 29 bp, Zea mays
tgcggtttcttttggcacaaatggcatga

SEQ ID NO: 42 - DNA, 30 bp, Zea mays
aaatctacctccaaccaacccagctttgta

SEQ ID NO: 43 - DNA, 30 bp, Zea mays
atcacaccaacttatcacctagaaaagcga

SEQ ID NO: 44 - DNA, 22 bp, Glycine max
ccttttgtctcccttttgtctc

SEQ ID NO: 45 - DNA, 28 bp, Oryza sativa
cgaggtgggcccgtaggtgggcccgtat

SEQ ID NO: 46 - DNA, 24 bp, Petroselinum crispum
tacctttttacccttcatgtcatc

SEQ ID NO: 47 - DNA, 25 bp, Pisum sativum
gtcgacaaaagttaggttagcaggc

SEQ ID NO: 48 - DNA, 21 bp, Hordeum vulgare
ggccgataacaaactccggcc

SEQ ID NO: 49 - DNA, 27 bp, Lycopersicon esculentum
ttttattcccaacaatagaaagtcttg

SEQ ID NO: 50 - DNA, 22 bp, Nicotiana tabacum
gatttggtcagaaagtcagtcc

SEQ ID NO: 51 - DNA, 31 bp, Triticum aestivum
gtagtgccaccaaacacaacataccaaatta

SEQ ID NO: 52 - DNA, 21 bp, Brassica napus
gatcccacatacacatacacg

SEQ ID NO: 53 - DNA, 27 bp, Helianthus annuus
cagctccaaatggtgatcttctcctgg

SEQ ID NO: 54 - DNA, 20 bp, Helianthus annuus
tatacagatgtagcatgtct

SEQ ID NO: 55 - DNA, 25 bp, Zea mays
ttgacgtgtaaagtaaatttacaac

SEQ ID NO: 56 - DNA, 22 bp, Pisum sativum
gacacgtagaatgagtcatcac

SEQ ID NO: 57 - DNA, 26 bp, Zea mays
gtccctctcccgtcccagagaaaccc

SEQ ID NO: 58 - DNA, 20 bp, Nicotiana tabacum
tgtcccccaacggtcttatt

SEQ ID NO: 59 - DNA, 20 bp, Arabidopsis thaliana
atatcataccgacatcagtt

SEQ ID NO: 60 - DNA, 20 bp, Arabidopsis thaliana
atatactaccgacatgagtt

SEQ ID NO: 61 - DNA, 31 bp, Arabidopsis thaliana
gataaagattacttcagatataacaaacgtt

SEQ ID NO: 62 - DNA, 23 bp, Nicotiana tabacum
ttcccctagctagatacttcatt

SEQ ID NO: 63 - DNA, 27 bp, Pisum sativum
cgattattgagatatataataaattag

SEQ ID NO: 64 - DNA, 21 bp, Lycopersicon esculentum
cgaaaacatacgcgcgaaatt

SEQ ID NO: 65 - DNA, 413 bp, Artificial Sequence
  synthetic
taggttaatttattgggcggtaattatagcttcgagaagaaccgagacgtggcgggctagcttcgagaagaaccgagacgtggcgggctagctaggttaattattggcgggtaattatagctccactgacgtaagggatgacgcacaattagctaggttaattattggcgataattatagctaggttaattattggcggtaattatagcatatcataccgacatcagtttagctaggttaattattggcggtaattatagcatatcataccgacatcagtttagcatatcataccgacatcagtttagctccactgacgtaagggatgacgcacaattagcatatcataccgacatcagtttagcatatcataccgacatcagtttagcttcgagaagaaccgagacgtggcg

SEQ ID NO: 66 - DNA, 392 bp, Artificial Sequence
  synthetic
gctaaactgatgtcggtatgatatgctagcccgccacgtctcggttcttctcgaagctaaactgatgtcggtatgatatgctaattgtgcgtcatcccttacgtcagtggagctagcccgccacgtctcggttcttctcgaagctaaactgatgtcggtatgatatgctataattaccgccaataattaacctagctaattgtgcgtcatcccttacgtcagtggagctaaactgatgtcggtagatatgctaatacgggcccacctacgggcccacctcggctaatacgggcccacctacgggcccacctcggctaaactgatgtcggtatgatatgctaattgtgcgtcatcccttacgtcagtggagctaaactgatgtcggtatgata

SEQ ID NO: 67 - DNA, 314 bp, Artificial Sequence
  synthetic
tagcatatcataccgacatcagtttagcatatcataccgacatcagtttagctccactgacgtaagggatgacgcacaattagccgaggtgggcccgtaggtgggcccgtattagcttcgagaagaaccgagacgtggcgggctagccgaggtgggcccgtaggtgggcccgtattagcttcgagaagaactgagacgtggcgggctagcatatcataccgacatcagtttagctaggttaattattggcggtaattatagctaggttaattattggcggtaattatagcttcgagaagaaccgaggacgtggc

SEQ ID NO: 68 - DNA, 278 bp, Artificial Sequence
  synthetic
tagcttcgagaagacgtggcgggccgccacgtctcggttcttctcgaagctataattaccgccaataattaacctagctataattaccgccaataattaacctagctataattaccgccaataattaacctagctaaactgatgtcggtatgatatgctaaactgatgtcggtatgatatgctaaactgatgtcggtatgatatgctaaactgatgtcggtatgatatgctagcccgccacgtctcggttcttctcgaagctaatacgggcccaccta

SEQ ID NO: 69 - DNA, 348 bp, Artificial Sequence
  synthetic
cgaggtgggcccgtaggtgggcccgtattagctccactgacgtaagggatgacgcacaattagctaggttaattattggcggtaattatagctccactgacgtaagggatgacgcacaattagcatatcataccgacatcagtttagctccactgacgtaagggatgacgcacaattagctccactgacgtaagggatgacgcacaattagccgaggtgggcccgtaggtgggcccgtattccactgacgtaagggatgacgcacaattagccgaggtgggcccgaggtgggcccgtattagcatatcataccgacatcagtttagcttcgagaagaaccgagtcgag

SEQ ID NO: 70 - DNA, 198 bp, Artificial Sequence
  synthetic
taaactgatgtcggtatgataatgccaacccggcaacgtcccggttcttctcgaagctataattaccgccaataattaacctagctaaactgatgtcggtatgatatgctaattgtgcgtcatcccttacgtcagtggagctaattgtgcgtcatcccttacgtcagtggagctccactgaacgtaagggatgacgtc

SEQ ID NO: 71 - DNA, 302 bp, Artificial Sequence
  synthetic
ttgtgcgtcatcccttacgtcagtggagtaattaccgccaataattaacctagctaaactgatgtcggtatgatatgctaaactgatgtcggtatgatatgctagcccgccacgtctcggttcttctcgaagctaatacgggcccacctacgggcccacctcggctaaactgatgtcggtatgatatgctaatacgggcccacctacgggcccacctcggctagcccgccacgtctcggttcttctcgaagctaaactgatgtcggtatgatatgctaaactgatgtcggtatgatatgcta

SEQ ID NO: 72 - DNA, 157 bp, Artificial Sequence
  synthetic
gtgcgtcatcccttacgtcagtggagcttcgagaagaaccgagacgtggcgggctagctaggttaattattggcggtaattatagctccactgacgtaagagcttcgagaagaaccgagacgtggcgggctagcatatcataccgacatcagtttag

(19) United States
(12) Patent Application Publication
(10) Pub. No.: US 2004/0016017 A1
(43) Pub. Date: Jan. 22, 2004

(54) Novel plant promoters and methods of use

(75) Inventors: Wesley B. Bruce, Grimes, IA (US); Xiping Niu, Johnston, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International, Inc.

(21) Appl. No.: 10/603,642

(22) Filed: Jun. 25, 2003

Related U.S. Application Data
(63) Continuation of application No. 09/766,399, filed on Jan. 19, 2001, now abandoned.
(60) Provisional application No. 60/177,437, filed on Jan. 21, 2000.

(51) Int. Cl. C07H 21/04; A01H 1/00; C12N 15/82

(52) U.S. Cl. 800/278; 536/23.6; 435/320.1; 435/468

(57) ABSTRACT

The present invention provides compositions and methods for regulating expression of heterologous nucleotide sequences in a plant. Compositions are novel nucleotide sequences for synthetic multimeric promoter element regions and plant promoters comprising the multimeric regions. Methods for expressing a heterologous nucleotide sequence in a plant using the promoter sequences disclosed herein are provided. The methods comprise transforming a plant cell with a heterologous nucleotide sequence operably linked to the promoters of the present invention and regenerating a stably transformed plant from the transformed plant cell.

### CROSS-REFERENCE TO RELATED APPLICATION

This application claims the benefit of, and hereby incorporates by reference, U.S. non-provisional application Ser. No. 09/766,399, filed Jan. 19, 2001, and U.S. provisional patent application No. 60/177,437, filed Jan. 21, 2000.

### FIELD OF THE INVENTION

The present invention relates to the field of plant molecular biology, more particularly to regulation of gene expression in plants.

### BACKGROUND OF THE INVENTION

Expression of heterologous DNA sequences in a plant host is dependent upon the presence of an operably linked promoter that is functional within the plant host. Choice of the promoter sequence will determine when and where within the organism the heterologous DNA sequence is expressed. Thus, where continuous expression is desired throughout the cells of a plant, constitutive promoters are utilized. In contrast, where gene expression in response to a stimulus is desired, inducible promoters are the regulatory element of choice. Where expression in particular organs is desired, tissue specific promoters are utilized. Additional regulatory sequences upstream and/or downstream from the core promoter sequence may be included in expression constructs of transformation vectors to bring about varying levels of constitutive or inducible expression of heterologous nucleotide sequences in a transgenic plant.

Frequently it is desirable to have constitutive expression of a DNA sequence throughout the cells of an organism. For example, increased resistance of a plant to infection by soil- and air-borne pathogens might be accomplished by genetic manipulation of the plant's genome to comprise a constitutive promoter operably linked to a heterologous pathogen-resistance gene such that pathogen-resistance proteins are continuously expressed throughout the plant's tissues.

Alternatively, it might be desirable to inhibit expression of a native DNA sequence within a plant's tissues to achieve a desired phenotype. In this case, such inhibition might be accomplished with transformation of the plant to comprise a constitutive promoter operably linked to an antisense nucleotide sequence, such that constitutive expression of the antisense sequence produces an RNA transcript that interferes with translation of the mRNA of the native DNA sequence.

Thus, isolation and characterization of promoters and promoter elements that can serve as regulatory regions for expression of heterologous nucleotide sequences of interest are needed for genetic manipulation of plants.

### SUMMARY OF THE INVENTION

Compositions and methods for regulating expression of heterologous nucleotide sequences in a plant are provided. The compositions comprise novel nucleotide sequences for synthetic multimeric promoter element regions (SMPERs) and plant promoters comprising the SMPERs. Particularly, plant promoters comprising one or more SMPERs that enhance expression directed by the promoter are provided.

Methods for expressing a heterologous nucleotide sequence in a plant using the promoter sequences disclosed herein are provided. The methods comprise transforming a plant cell with a transformation vector that comprises a heterologous nucleotide sequence operably linked to one of the plant promoters of the present invention and regenerating a stably transformed plant from the transformed plant cell. In this manner, expression levels in a plant cell, plant organ, plant tissue or plant seed can be controlled.

### BRIEF DESCRIPTION OF THE DRAWINGS

FIG. 1 shows the sequences of 64 defined or putative promoter elements or transcription factor binding sites. Promoter elements selected for synthesis of SMPERs are designated by an asterisk.

FIG. 2 shows the 2-dimensional (8×8) register for pooling of transcription factor binding sites and/or promoter elements Em1a (SEQ ID NO.: 1), ABRE1 (SEQ ID NO.: 2), ABRE A (SEQ ID NO.: 3), Prolamin P-box (SEQ ID NO.: 4), Z2 and Z3 box (SEQ ID NO.: 5), 35S AS-2 (SEQ ID NO.: 6), 35S AS-1 (SEQ ID NO.: 7), OCS ele (SEQ ID NO.: 8), GCC-box (SEQ ID NO.: 9), GH3 D1 (SEQ ID NO.: 10), GH3 D3 (SEQ ID NO.: 11), P3 (SEQ ID NO.: 12), GT-1 rbcS3A (SEQ ID NO.: 13), TCA motif (SEQ ID NO.: 14), C-repeat/DRE (SEQ ID NO.: 15), HSE (SEQ ID NO.: 16), ERE (SEQ ID NO.: 17), gln2 PR box (SEQ ID NO.: 18), HBP-1a (SEQ ID NO.: 19), A1 PROMOTER (SEQ ID NO.: 20), Bz1 PROMOTER (SEQ ID NO.: 21), CHS promoter (SEQ ID NO.: 22), Boxll (SEQ ID NO.: 23), phyA GT-2 (SEQ ID NO.: 24), GT-2 like (SEQ ID NO.: 25), Phy PF1 (SEQ ID NO.: 26), AT-com (SEQ ID NO.: 27), AG site (SEQ ID NO.: 28), AP3 site (SEQ ID NO.: 29), TGAC motif (SEQ ID NO.: 30), CAGT motif (SEQ ID NO.: 31), Dof1/Dof2 (SEQ ID NO.: 32), pr2 oligomer 11 (SEQ ID NO.: 33), CE1 (SEQ ID NO.: 34), H-box1 (SEQ ID NO.: 35), H-box2 (SEQ ID NO.: 36), lox1 (SEQ ID NO.: 37), PR-2d (SEQ ID NO.: 38), ROL6 (SEQ ID NO.: 39), SGB box 2/3 (SEQ ID NO.: 40), SGB box 6-8 (SEQ ID NO.: 41), MS-BS7 box 1-3 (SEQ ID NO.: 42), MS-BS7 box 22-24 (SEQ ID NO.: 43), AuxRE DR5 (SEQ ID NO.: 44), PCNA IIA (SEQ ID NO.: 45), PAL1 Box E (SEQ ID NO.: 46), myb26 (SEQ ID NO.: 47), GARE (SEQ ID NO.: 48), E8 (SEQ ID NO.: 49), E1RE (SEQ ID NO.: 50), CA (SEQ ID NO.: 51), napA (SEQ ID NO.: 52), HaG3-A-75 (SEQ ID NO.: 53), HaG3-A-111 (SEQ ID NO.: 54), Prolamin box (SEQ ID NO.: 55), TGAC-like (SEQ ID NO.: 56), SP20+6 (SEQ ID NO.: 57), MSA RT1 (SEQ ID NO.: 58), DRE rd29A1 (SEQ ID NO.: 59), DRE rd29A2 (SEQ ID NO.: 60), CGF-1 (SEQ ID NO.: 61), Itp1 D1 (SEQ ID NO.: 62), ENBP1 (SEQ ID NO.: 63), and MRE (SEQ ID NO.: 64).

FIG. 3 depicts promoter elements characterized as having strong binding to maize nuclear extracts. These promoter elements are shaded.

FIG. 4 is a schematic representation of the expression cassette in the Adhl intron plus expression vector, comprising specific synthetic multimeric promoter element regions. The A designations (e.g. A18) refer to clone numbers comprising the particular SMPER depicted. Arrows indicate the orientation of each promoter element. Identity of each promoter element is shown by the key at the bottom. LexA depicts the negative control. “+++” indicates high enhancer activity.

FIG. 5 is a schematic representation of the expression cassette in the Adhl intron minus expression vector, comprising specific synthetic multimeric promoter element regions. The A designations (e.g. A42) refer to clone numbers comprising the particular SMPER depicted. Arrows indicate the orientation of each promoter element. Identity of each promoter element is shown by the key at the bottom. LexA depicts the negative control. “+++” indicates high enhancer activity.

FIGS. 6a, 6b, 6c, and 6d depict results of transient assays for luciferase activity in extracts of maize seedlings transformed with the indicated SMPER constructs.

FIGS. 7-14 provide the respective nucleotide sequences for SMPER A15 (SEQ ID NO. 65), A18 (SEQ ID NO. 66), A23 (SEQ ID NO. 67), A24 (SEQ ID NO. 68), A42 (SEQ ID NO. 69), A44 (SEQ ID NO. 70), A48 (SEQ ID NO. 71), and A51 (SEQ ID NO. 72), respectively. Spacer sequences are designated by underscoring. Individual promoter elements are designated according to the corresponding element names shown in FIG. 1.

### DETAILED DESCRIPTION OF THE INVENTION

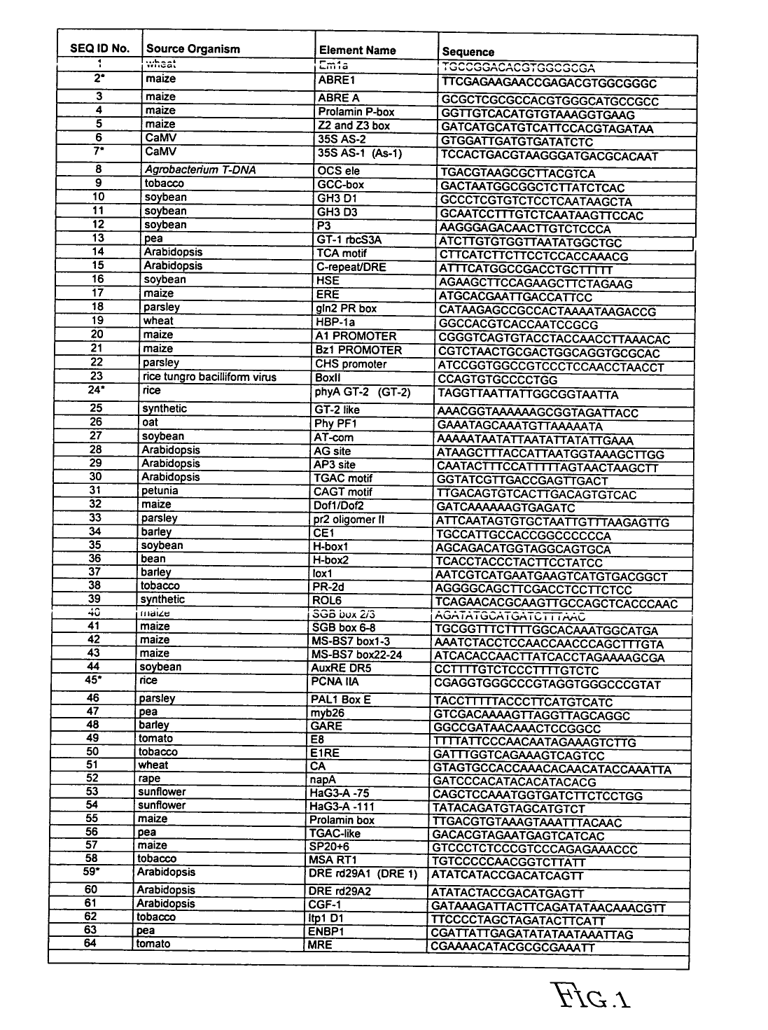

Compositions of the present invention are directed to novel nucleotide sequences for synthetic multimeric promoter element regions (SMPERs) and plant promoters comprising the SMPERs. Particularly, plant promoters are provided comprising at least one SMPER that enhances transcription directed by the promoter. The SMPERs comprise novel arrangements of individual promoter elements. See, for example, FIG. 1. In particular, specific combinations comprising the promoter elements PCNA IIA, GT-2, ABRE 1, As-1 and DRE 1 are provided.

The multimeric promoter element regions of the invention, and the plant promoters of the invention comprising the multimeric promoter element regions, are synthetic. By “synthetic” is intended that nucleotide sequences of the multimeric promoter element regions of the invention, or that of the plant promoters of the invention comprising the multimeric promoter element regions, are not found in nature.

By “synthetic multimeric promoter element region” or “SMPER” is intended a nucleic acid having a nucleotide sequence comprising more than one promoter element, wherein the arrangement of the multimeric combination of the promoter elements is not found in nature. That is, the invention recognizes that the promoter elements can be provided in any sequence or arrangement to provide a SMPER. Such SMPER is then tested for its effect on transcription. It is recognized that the elements may be presented in any order. In some instances, elements may be duplicated, i.e., more than one copy of an individual element may be present in the SMPER. Using the methods described herein, combinations of promoter elements can be tested for their effect on transcription. In this manner, any combination is encompassed by the invention. Preferred SMPERs of the invention comprise promoter elements including but not limited to PCNA IIA, GT-2, ABRE 1, As-1 and DRE1.

The SMPERs of the invention may be used with any promoter, native or synthetic. More particularly, the SMPERs may be used with any plant promoter, native or synthetic. By “plant promoter” is intended a promoter capable of driving expression in a plant cell.

In reference to a promoter, by “native” is intended a promoter capable of driving expression in a particular cell, wherein the nucleotide sequence of the promoter is found in that cell in nature. That is, the nucleotide sequence of a native promoter can be isolated from the cell, or the corresponding cell source, without introduction of the promoter to the cell, the cell source, or an ancestor thereof. By “cell source” is intended an organism or tissue from which the cell is derived.

In reference to a promoter, by “synthetic” is intended a promoter capable of driving expression in a particular cell, wherein the nucleotide sequence of the promoter is not found in nature. That is, the nucleotide sequence of a synthetic promoter cannot be isolated from the cell, or the corresponding cell source, without having introduced the promoter to the cell, the cell source, or an ancestor thereof. Thus, the combination of the promoters and the SMPERs are synthetic. These combinations are not found in nature and cannot be isolated from a native plant, plant cell, or plant tissue.

The individual promoter elements PCNA IIA, GT-2, ABRE1, As-1 and DRE1 are described in FIG. 1. The methods for synthesizing and isolating the plant promoters of the invention are provided in the Examples described below.

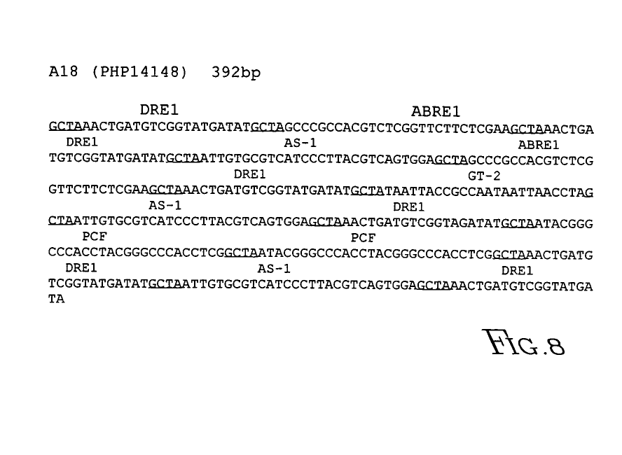

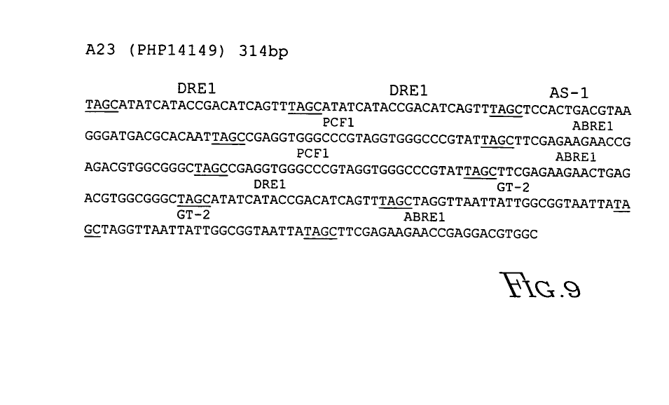

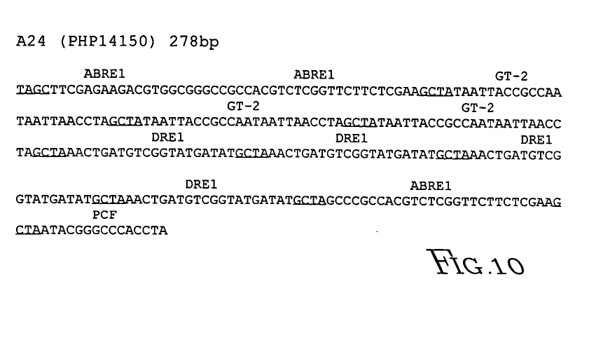

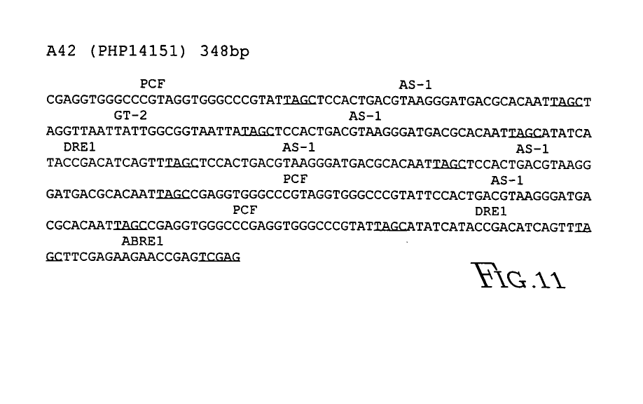

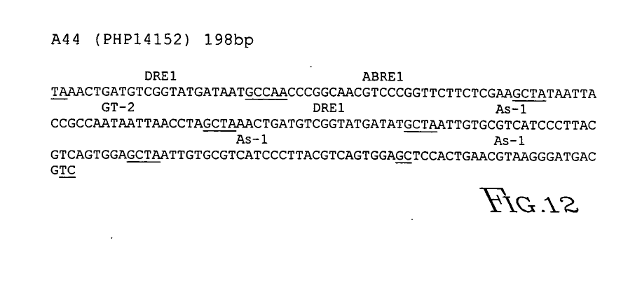

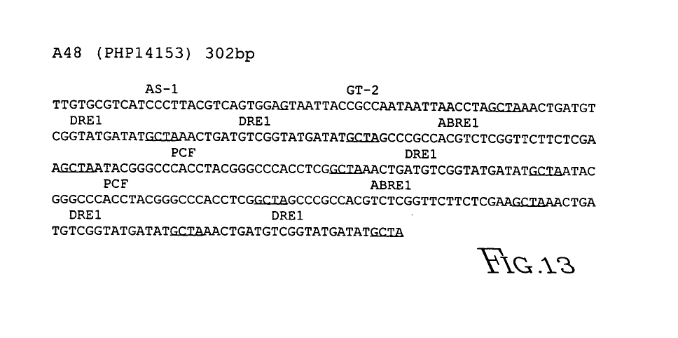

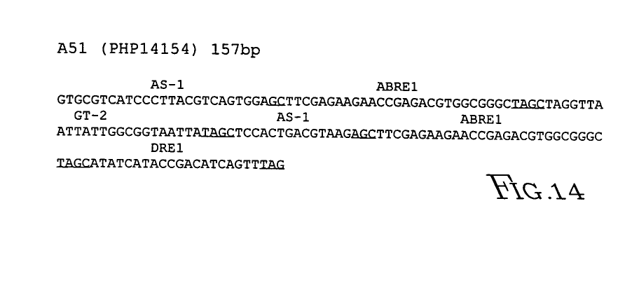

The nucleotide sequences of SMPERs of the present invention comprising specific combinations of promoter elements PCNA IIA, GT-2, ABRE1, As-1 and DRE1 are set forth in FIGS. 7-14 (SEQ ID NOS: 65-72).

The invention encompasses isolated or substantially purified nucleic acid compositions. An “isolated” or “purified” nucleic acid molecule, or biologically active portion thereof, is substantially free of other cellular material, or culture medium when produced by recombinant techniques, or substantially free of chemical precursors or other chemicals when chemically synthesized.

As used herein, the term “plant” includes reference to whole plants and their progeny; plant cells; plant parts or organs, such as embryos, pollen, ovules, seeds, flowers, kernels, ears, cobs, leaves, husks, stalks, stems, roots, root tips, anthers, silk and the like. Plant cell, as used herein, further includes, without limitation, cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. The class of plants which can be used in the methods of the invention is generally as broad as the class of higher plants amenable to transformation techniques, including both monocotyledonous and dicotyledonous plants. A particularly preferred plant is Zea mays.

By “promoter” or “transcriptional initiation region” is intended a regulatory region of DNA usually comprising a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, and referred to as promoter elements which influence the sequences for the promoter regions disclosed herein. Promoter elements located upstream or 5′ to the TATA box are also referred to as upstream promoter elements. In particular embodiments of the invention, the SMPERs of the invention are positioned upstream or 5′ to the TATA box. However, the invention also encompasses plant promoter configurations in which the SMPERs are positioned downstream or 3′ to the TATA box.

The promoter elements of the invention may act as enhancers or suppressors of expression.

Enhancers are nucleotide sequences that act to enhance or increase the expression directed by a promoter region. An enhancer can be identified by comparing the expression level directed by a sample promoter comprising the enhancer sequence to be tested placed at any position upstream or downstream of the promoter, relative to a control promoter that does not comprise the sequence in question. Known individual enhancer elements for plants include, for example, the SV40 enhancer region, the 35S enhancer element, and the like. The SMPERs of the invention enhance expression of coding sequences operably linked to the plant promoters comprising the SMPERs. Accordingly, the SMPERs of the invention may act as enhancers. Li et al. (1999), Nature Biotechnology: 17: 241-245, describe random assembly of muscle promoter elements for achieving enhanced promoter activity in muscle.

By “suppressors” are intended nucleotide sequences that mediate suppression or decrease in the expression directed by a promoter region. That is, suppressors are the DNA sites through which transcription repressor proteins exert their effects. Suppressors can mediate suppression of expression by overlapping transcription start sites or transcription activator sites, or they can mediate suppression from distinct locations with respect to these sites. The SMPERs of the invention may act as suppressors.

The SMPERs may be operably linked to any promoter of interest. While not a limitation, it may be preferable to use core promoters. By “core promoter” is intended a promoter without regulatory promoter elements such as enhancers, suppressors, and the like. Promoters of interest include but are not limited to constitutive, weak, pathogen-indicible, wound-inducible, chemical-regulated, and tissue-specific promoters, including but not limited to leaf-specific, root-specific, and seed-specific promoters.

Such constitutive promoters include, for example, the core promoter of the Rsyn7 (U.S. Pat. No. 6,072,050); the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; and 5,608,142.

See also the copending application entitled “Constitutive Maize Promoters”, U.S. application Ser. No. 09/257,584, filed Feb. 25, 1999, and herein incorporated by reference.

Such pathogen-inducible promoters include but are not limited to those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also the copending application entitled “Inducible Maize Promoters”, U.S. application Ser. No. 09/257,583, filed Feb. 25, 1999, herein incorporated by reference. Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet 2:93-98; and Yang (1996) Proc. Natl. Acad. Sci. USA 93:14972-14977. See also, Chen et a. (1996) Plant J. 10:955-966; Zhang et al. (1994) Proc. Natl. Acad. Sci. USA 91:2507-2511; Warner et al (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Such wound-inducible promoters include but are not limited to potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Such chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Such tissue-preferred promoters include but are not limited to Yamamoto et al. (1997) Plant J. 12(2)255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3): 1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6): 1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-specific promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol Biol. 23(6):1129-1138; and Matsuoka et al. (1993) Proc. Natl. Acad. Sci. USA 90(20):9586-9590.

Root-specific promoters are known and can be selected from the many available in the literature or isolated de novo from various compatible species. See, for example, Hire et al. (1992) Plant Mol. Biol. 20(2): 207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus comiculatus, and in both instances root-specific promoter activity was preserved. Leach and Aoyagi (1991) describe their analysis of the promoters of the highly expressed rolC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptll (neomycin phosphotransferase 11) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691). See also U.S. Pat. Nos. 5,837,876; 5,750,386; 5,633,363; 5,459,252; 5,401,836; 5,110,732; and 5,023,179.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase); and celA (cellulose synthase) (see the copending application entitled “Seed-Preferred Promoters,” U.S. application Ser. No. 09/377,648, filed Aug. 19, 1999, herein incorporated by reference). Gama-zein is a preferred endosperm-specific promoter. Glob-1 is a preferred embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc.

Generally, the plant promoter sequences of the present invention, when operably linked to a heterologous nucleotide sequence of interest and inserted into a transformation vector, control constitutive expression of the heterologous nucleotide sequence in the cells of a plant stably transformed with this vector. By “constitutive” is intended expression in the cells throughout a plant at most times and in most tissues. It is recognized that depending on the particular host plant or tissue, the particular SMPER or promoter comprising the SMPERs, and variants and fragments thereof, could be used to control tissue-preferred or tissue-specific expression.

By “heterologous nucleotide sequence” is intended a sequence that is not naturally occurring with the promoter sequence. The SMPERs and the plant promoters of the invention comprising the SMPERs are not found in nature. Therefore, any sequence of interest operably linked to a promoter comprising the SMPERs of the invention is a heterologous nucleotide sequence. While this linked nucleotide sequence is heterologous to the promoter sequence, it may be homologous (native) or heterologous (foreign) to the plant host.

The isolated SMPER sequences of the present invention, and plant promoter sequences comprising the SMPERs, can be modified to provide for a range of expression levels of the heterologous nucleotide sequence. Thus, less than the entire promoter regions may be utilized and the ability to drive expression of the coding sequence retained. However, it is recognized that expression levels of the mRNA may be decreased with deletions of portions of the promoter sequences. Likewise, the general nature of expression may be changed.

Modifications of the SMPER sequences of the present invention and of plant promoter sequences comprising the SMPERs can provide for a range of expression. Thus, they may be modified to be weak promoters or strong promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

The nucleotide sequences for the plant promoters of the present invention may comprise the sequences set forth in FIGS. 7-14 (SEQ ID NO: 65-72) or any sequence having substantial identity to the sequences. By “substantial identity” is intended a sequence exhibiting substantial functional and structural equivalence with the sequence set forth. Any functional or structural differences between substantially identical sequences do not affect the ability of the sequence to function as a promoter as disclosed in the present invention. Thus, the plant promoter of the present invention will direct enhanced expression of an operably linked heterologous nucleotide sequence. Two SMPER nucleotide sequences are considered substantially identical when they have at least about 80%, preferably at least about 85%, more preferably at least about 90%, still more preferably at least about 95%, and most preferably at least about 98% sequence identity.

Fragments and variants of the SMPER nucleotide sequences set forth herein are also encompassed by the present invention. By “fragment” is intended a portion of the nucleotide sequence that is longer than the shortest individual promoter element contained in the particular portion. Fragments of a nucleotide sequence may retain biological activity and hence enhance expression of a nucleotide sequence operably linked to a synthetic promoter comprising the SMPER. (See Lam et al. (1989) Proc. Natl. Acad. Sci. USA 86:7890; See also Oliphant et al (1989) Mol. Cell Biol. 9: 2944-2949; Niu and Guiltinan (1994) Nucleic Acid Res. 22: 4969-497; Oeda, et al EMBO J. 10:1793; and Catron et al. (1993) Mol. Cell Biol. 13: 2354-2365.) Alternatively, fragments of a nucleotide sequence that are useful as hybridization probes or PCR primers generally do not retain biological activity. Thus, fragments of a nucleotide sequence may range from at least 7 to 10, or about 21, 25, 28, or 29 nucleotides, about 50 nucleotides, about 100 nucleotides, and up to the full length of a nucleotide sequence of the invention.

A biologically active portion of a promoter comprising the SMPERs of the invention can be prepared by synthesizing a portion of one of the promoter nucleotide sequences and assessing the activity of the fragment. Nucleic acid molecules that are fragments of a promoter nucleotide sequence comprise at least 21, 50, 75, 100, 150, or 200 nucleotides, or up to the number of nucleotides present in a full-length promoter nucleotide sequence disclosed herein (for example, 413, 392, 314, 278, 348,198, 302, or 157 nucleotides for FIGS. 7, 8, 9, 10, 11, 12, 13, or 14 (SEQ ID NO: 65-72), respectively).

Variants of these promoter fragments, such as those resulting from site-directed mutagenesis, are encompassed by the compositions of the present invention.

The invention encompasses variants of the SMPERs and of the plant promoter sequences comprising the SMPERs. By “variants” is intended substantially identical sequences. Naturally occurring variants of the individual promoter element sequences can be identified and/or isolated with the use of well-known molecular biology techniques, as, for example, with PCR and hybridization techniques as outlined below. The invention encompasses variants of the SMPERs and plant promoter sequences disclosed herein in which one or more of the individual promoter elements are substituted by a natural variant of that element. For example, and without limitation, the element ABRE 1 could be substituted by the ABRE A; and/or DRE1 could be substituted by DRE2.

The invention encompasses variants of the SMPERs and plant promoter sequences disclosed herein in which one or more of the individual promoter elements is in the alternative orientation. By “orientation” is intended the 5′ to 3′ (sense) or the 3′ to 5′ (antisense) configuration of a promoter element sequence contained in a contiguous strand, relative to the configuration of other promoter elements and/or the TATA box contained in that strand.

The invention encompasses plant promoter and SMPER sequences in which the individual promoter elements are separated and/or flanked by spacer sequences. By “spacer sequence” is intended the nucleotide sequence contained in an SMPER that is not a promoter element. The invention also encompasses variants of the SMPERs and plant promoter sequences comprising contiguous multimers of individual promoter elements thereby containing no spacer sequences; variants in which one or more individual elements are separated or flanked by spacer sequences, and variants comprising spacer sequences that are different than the spacer sequences disclosed herein.

Variant SMPER and promoter nucleotide sequences include synthetically derived nucleotide sequences, such as those generated, for example, by using site-directed mutagenesis, but which still exhibit promoter activity. Methods for mutagenesis and nucleotide sequence alterations are well known in the art. See, for example, Kunkel (1985) Proc. Natl. Acad. Sci. USA 82:488492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Generally, a nucleotide sequence of the invention will have at least 80%, preferably 85%, 90%, 95%, up to 98% or more sequence identity to its respective reference promoter nucleotide sequence, and enhance or promote expression of heterologous coding sequences in plants or plant cells.

Variant promoter nucleotide sequences also encompass sequences derived from a mutagenic and recombinogenic procedure such as DNA shuffling. With such a procedure, one or more different promoter sequences can be manipulated to create a new promoter possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Biologically active variants of the promoter sequences should retain promoter activity and thus promote or enhance expression of an operably linked heterologous nucleotide sequence. Promoter activity may be measured by Northern blot analysis. See, for example, Sambrook et al. (1989) Molecular Cloning: A Laboratory Manual (2d ed., Cold Spring Harbor Laboratory Press, Plainview, N.Y.), herein incorporated by reference. Protein expression indicative of promoter activity can be measured by determining the activity of a protein encoded by the coding sequence operably linked to the particular promoter, including but not limited to such examples as GUS (b-glucoronidase; Jefferson (1987) Plant Mol. Biol. Rep. 5:387), GFP (green florescence protein; Chalfie et al. (1994) Science 263:802), luciferase (Riggs et al. (1987) Nucleic Acids Res. 15(19):8115 and Luehrsen et al. (1992) Methods Enszymol. 216:397414), and the maize genes encoding for anthocyanin production (Ludwig et al. (1990) Science 247:449).

It is recognized that the SMPERs are not found in nature. That is, the combination of the individual promoter elements is novel. However, it is also recognized that the nucleotide sequences of the invention may be used to isolate substantially identical sequence fragments from natural sources, particularly plants. In this manner, methods such as PCR, hybridization, and the like can be used to identify such sequences based on their sequence homology to the sequences set forth herein. Such methods are generally known in the art and are disclosed in, for example, Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Sequences isolated based on their sequence identity to a fragment of the sequences set forth herein are encompassed by the present invention. Such individual elements can be used in a SMPER.

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of identity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl 1% SDS (sodium dodecyl sulphate) at 37° C., and a wash in 1× to 2× SSC (20×SSC=3.0 M NaCl/0.3 M trisodium citrate) at 50 to 55° C. Exemplary moderate stringency conditions include hybridization in 40 to 45% formamide, 1.0 M NaCl,1% SDS at 37° C., and a wash in 0.5× to 1× SSC at 55 to 60° C. Exemplary high stringency conditions include hybridization in 50% formamide, 1 M NaCl, 1% SDS at 37° C., and a wash in 0.1× SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6(log M)+0.41(% GC)−0.61(% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, N.Y.). See Sambrook et al. (1989) Molecular Cloning: A Laboratory Manual (2d ed., Cold Spring Harbor Laboratory Press, Plainview, N.Y.).

In general, sequences that have promoter or enhancer activity and hybridize to the sequences disclosed herein will be at least 80%, 85%, 90%, 95% to 98% or more identical with the disclosed sequences.

Methods of alignment to determine the extent of identity of two sequences are well known in the art. Thus, the determination of percent identity between any two sequences can be accomplished using a mathematical algorithm. Preferred, non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG® programs (Accelrys, Inc., San Diego, Calif.)); and Sequencher (GeneCodes, Ann Arbor, Mich.). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. Alignment may also be performed manually by inspection. For purposes of the present invention, comparison of nucleotide or protein sequences for determination of percent sequence identity to the sequences disclosed herein is preferably made using GAP (GCG Version 10) with its default parameters, or any equivalent sequence comparison program. By “equivalent program” is intended any sequence comparison program that, for any two sequences in question, generates an alignment having substantially identical nucleotide or amino acid residue matches and a substantially identical percent sequence identity when compared to the corresponding alignment generated by the preferred program.

As used herein, “sequence identity” or “identity” in the context of two nucleic acid sequences makes reference to the residues in the two sequences that are the same when aligned for maximum correspondence over a specified comparison window.

The term “substantial identity” of polynucleotide sequences means that a polynucleotide comprises a sequence that has at least 80% sequence identity, preferably at least 85%, more preferably at least 90%, even more preferably at least 95%, and most preferably at least 98%, compared to a sequence of the invention using one of the alignment programs described above using standard or default parameters.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. The nucleotide sequences for the SMPERs and promoters of the present invention, as well as variants and fragments thereof, are useful in the genetic manipulation of any plant when operably linked with a heterologous nucleotide sequence whose expression is to be controlled to achieve a desired phenotypic response. By “operably linked” is intended that the transcription or translation of the heterologous nucleotide sequence is under the influence of the promoter sequence. In this manner, the nucleotide sequences for the promoters of the invention are provided in expression cassettes along with nucleotide sequences of interest for expression in the plant of interest.

Such DNA constructs or expression cassettes will comprise a transcriptional initiation region comprising one of the promoter nucleotide sequences of the present invention, or variants or fragments thereof, operably linked to the heterologous nucleotide sequence whose expression is to be controlled by the promoters disclosed herein. Such an expression cassette is provided with a plurality of restriction sites for insertion of the nucleotide sequence to be under the transcriptional regulation of the regulatory regions. The expression cassette may additionally contain selectable marker genes.

The transcriptional cassette will include in the 5′-to-3′ direction of transcription, a transcriptional and translational initiation region, a heterologous nucleotide sequence of interest, and a transcriptional and translational termination region functional in plant cells. The termination region may be native with the transcriptional initiation region comprising one of the promoter nucleotide sequences of the present invention, may be native with the DNA sequence of interest, or may be derived from another source. Convenient termination regions are available from the Ti-plasmid of A. tumefaciens, such as the octopine synthase and nopaline synthase termination regions. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. 1989) Nucleic Acids Res. 17:7891-7903; Joshi et al. (1987) Nucleic Acid Res. 15:9627-9639.

The expression cassette comprising the promoter sequence of the present invention operably linked to a heterologous nucleotide sequence may also contain at least one additional nucleotide sequence for a gene to be cotransformed into the organism. Alternatively, the additional sequence(s) can be provided on another expression cassette. Where appropriate, the heterologous nucleotide sequence whose expression is to be under the control of the promoter sequence of the present invention and any additional nucleotide sequence(s) may be optimized for increased expression in the transformed plant. That is, these nucleotide sequences can be synthesized using plant-preferred codons for improved expression. Methods are available in the art for synthesizing plant-preferred nucleotide sequences. See, for example, U.S. Pat. Nos. 5,380,831 and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the heterologous nucleotide sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 540 noncoding region) (Elroy-Stein et al. (1989) Proc. Nat. Acad. Sci. USA 86:6126-6130); potyvirus leaders, for example, TEV leader (Tobacco Etch Virus) (Allison et al. (1986)); MDMV leader (Maize Dwarf Mosaic Virus) (Virology 154:9-20); human immunoglobulin heavy-chain binding protein (BiP) (Macejak and Sarnow (1991) Nature 353:90-94); untranslated leader from the coat protein mRNA of alfalfa mosaic virus (AMV RNA 4) (Jobling and Gehrke (1987) Nature 325:622-625); tobacco mosaic virus leader (TMV) (Gallie et al. (1989) Molecular Biology of RNA, pages 237-256); and maize chlorotic mottle virus leader (MCMV) (Lommel et al. (1991) Virology 81:382-385). See also Della-Cioppa et al. (1987) Plant Physiology 84:965-968. Other methods known to enhance translation and/or mRNA stability can also be utilized, for example, introns, and the like.

In those instances where it is desirable to have the expressed product of the heterologous nucleotide sequence directed to a particular organelle, such as the chloroplast or mitochondrion, or secreted at the cell's surface or extracellularly, the expression cassette may further comprise a coding sequence for a transit peptide. Such transit peptides are well known in the art and include, but are not limited to, the transit peptide for the acyl carrier protein, the small subunit of RUBISCO, plant EPSP synthase, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments, or other manipulations may be involved to provide for convenient restriction sites, removal of superfluous DNA, removal of restriction sites, or the like. For this purpose, in vitro mutagenesis, primer repair, restriction, annealing, resubstitutions, for example, transitions and transversions, may be involved.

The promoters may be used to drive reporter genes or selectable marker genes. Examples of suitable reporter genes known in the art can be found in, for example, Jefferson et al. (1991) in Plant Molecular Biology Manual, ed. Gelvin et al. (Kluwer Academic Publishers), pp.1-33; DeWet et al. (1987) Mol. Cell. Biol. 7:725-737; Goff et al. (1990) EMBO J. 9:2517-2522; and Kain et al. (1995) BioTechniques 19:650-655; and Chiu et al. (1996) Current Biology 6:325-330.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella et al. (1983) EMBO J. 2:987-992); methotrexate (Herrera Estrella et al. (1983) Nature 303:209-213; Meijer et al. (1991) Plant Mol. Biol. 16:807-820); hygromycin (Waldron et al. (1985) Plant Mol. Biol. 5:103-108; Zhijian et al (1995) Plant Science 108:219-227); streptomycin (Jones et al. (1987) Mol. Gen. Genet. 210:86-91); spectinomycin (Bretagne-Sagnard et al. (1996) Transgenic Res. 5:131-137); bleomycin (Hille et al. (1990) Plant Mol. Biol. 7:171-176); sufonamide (Guerineau et al. (1990) Plant Mol. Biol. 15:127-136); bromoxynil (Stalker et al. (1988) Science 242:419-423); glyphosate (Shaw et al. (1986) Science 233:478-481); phosphinothricin (DeBlock et al. (1987) EMBO J. 6:2513-2518).

Other genes that could serve utility in the recovery of transgenic events but might not be required in the final product would include, but are not limited to, such examples as GUS (b-glucoronidase; Jefferson (1987) Plant Mol. Biol. Rep. 5:387), GFP (green florescence protein; Chalfie et al. (1994) Science 263:802), luciferase (Riggs et al. (1987) Nucleic Acids Res. 15(19):8115 and Luehrsen et al. (1992) Methods Enszymol. 216:397-414), and the maize genes encoding for anthocyanin production (Ludwig et al. (1990) Science 247:449).

The expression cassette comprising the particular promoter sequence of the present invention operably linked to a heterologous nucleotide sequence of interest can be used to transform any plant. In this manner, genetically modified plants, plant cells, plant tissue, seed, and the like can be obtained. Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055), direct gene transfer (Paszkowski et al. (1984) EMBO J. 3:2717-2722), and ballistic particle acceleration (see, for example, Sanford et al., U.S. Pat. No. 4,945,050; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:43054309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp.197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested, to ensure expression of the desired phenotypic characteristic has been achieved.

The present invention may be used for transformation of any plant species, including, but not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena sativa), barley (Hordeum vulgare), vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotil), ponderosa pine (Pinus ponderosa), lodgepole pine (Pinus contorta), and Monterey pine (Pinus radiata); Douglas-fir (Pseudotsuga menziesil); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

Plants of particular interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The promoter sequences and methods disclosed herein are useful in regulating expression of any heterologous nucleotide sequence in a host plant. Thus, the heterologous nucleotide sequence operably linked to the promoters disclosed herein may be a structural gene encoding a protein of interest. Examples of such heterologous genes include, but are not limited to, genes encoding proteins conferring resistance to abiotic stress, such as drought, temperature, salinity, and toxins such as pesticides and herbicides, or to biotic stress, such as attacks by fungi, viruses, bacteria, insects, and nematodes, and development of diseases associated with these organisms.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increases, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

Agronomically important traits such as oil, starch, and protein content can be genetically altered in addition to using traditional breeding methods. The quality of grain is reflected in traits such as levels and types of oils, saturated and unsaturated, quality and quantity of essential amino acids, and levels of cellulose. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,990,389, 5,885,801, and 5,885,802, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and PCT Publication No. WO98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,737,514; 5,723,756; 5,593,881; and Geiser et al. (1986) Gene 48:109); lectins (Van Damme et al. (1994) Plant Mol. Biol. 24:825); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptll gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

Commercial traits can also be encoded on a gene or genes that could increase, for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxybutyrate synthase), and acetoacetyl-CoA reductase (see Schubert et al. (1988) J. Bacteiol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

Alternatively, the heterologous nucleotide sequence operably linked to one of the promoters disclosed herein may be an antisense sequence for a targeted gene. Thus, sequences can be constructed which are complementary to, and will hybridize with, the messenger RNA (mRNA) of the targeted gene. Modifications of the antisense sequences may be made, as long as the sequences hybridize to and interfere with expression of the corresponding mRNA. In this manner, antisense constructions having 70%, preferably 80%, more preferably 85% sequence similarity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used. When delivered into a plant cell, expression of the antisense DNA sequence prevents normal expression of the DNA nucleotide sequence for the targeted gene. In this manner, production of the native protein encoded by the targeted gene is inhibited to achieve a desired phenotypic response. Thus the promoter is linked to antisense DNA sequences to reduce or inhibit expression of a native protein in the plant.

The following examples are offered by way of illustration and not by way of limitation.

### EXPERIMENTAL

### EXAMPLE 1

### Collection and Identification of Promoter Elements

Sequences of 64 defined or putative promoter elements or transcription factor binding sites were collected, each element 20-40 base pairs(bp) long. The sequences are shown in FIG. 1 in the 5′ to 3′ (sense) direction. Oligonucleotides (oligos) corresponding to the top (sense) strands and bottom (antisense) strands of these promoter element sequences were synthesized by automated DNA synthesizer. For DNA synthesis, the spacer sequence TAGC was added to all the top strand oligos and GCTA to all the bottom strand oligos to facilitate subsequent DNA manipulation.

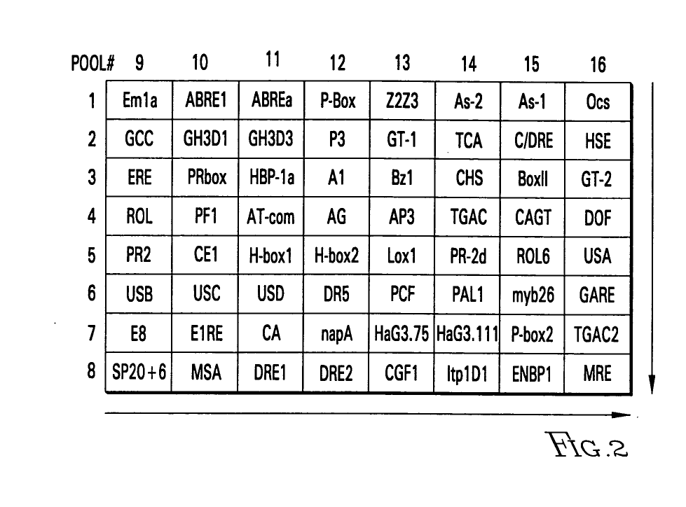

The 64 pairs of corresponding sense and antisense synthesized oligos were annealed in individual reactions (88° C. for 2 minutes (min.), 65° C. for 15 min., 37° C. for 15 min., 25° C. for 5 min.). Thereafter, the oligos were arranged and registered in an 8×8 format in a microtiter plate. These oligos were pooled using a 2-dimensional pooling strategy (8 horizontal and 8 vertical pools). Each pool contains 8 oligo pairs, as indicated in FIG. 2. The 16 pools of oligos were labeled with Klenow enzyme in the presence of P-32-dCTP in separate reactions (200 ng DNA in each 20 μl reaction). The labeled DNA was purified by a spin column (Bio-Gel P-6 spin column, Biorad). These DNA probes were used in DNA-binding reactions with maize nuclear extracts.

Nuclear extracts were prepared using a protocol modified from Green et al. (1988) “In vitro DNA Footprinting,” in Plant Molecular Biology Manual, ed. Gelvin, Schilperoort, and Verma (Kluwer Academic Publishers, Dordrecht) B11: 1-22. Seeds were germinated in the dark at 24° C. Roots from 4-day seedlings were collected and 4× volume of the Homogenizing Buffer (25 mM Hepes/KOH pH 7.6, 10 mM MgCl2, 0.3 M sucrose, 0.5% Triton X-100, 5 mM β-mercaptoethanol, 1 mM PMSF) was added. Tissues were dissected into small pieces using a commercial Waring blender at low speed for 10 sec and ground to paste with mortar and pestle. Homogenized tissues were filtered through two layers of miracloth (CalBiochem) and one layer of 70 μm nylon screen. The extracts were centrifuged in a Sorval GSA rotor, 4500 rpm, 15 minutes. Nuclei pellets were then resuspended gently with a paint brush in Homogenizing Buffer and centrifuged as above. This step was repeated once. After the last centrifugation, nuclei were resuspended in Nuclear Lysis Buffer (15 mM Hepes/KOH pH 7.6, 110 mM KCl, 5 mM MgCl2, 1 mM DTT, 1 mM PMSF, 5 μg/ml leupeptin, 2 μg/ml aprotinin, 1 μg/ml pepstatin A). NaCl was added in a dropwise manner to a final concentration of 0.5 M. Nuclear proteins were extracted from the nuclei by incubation of the NaCl mixture on ice for 40 minutes with gentle shaking. The extract was centrifuged in Sorval SS34 rotor, 16K rpm, for 30 minutes. Supernatants were frozen in liquid nitrogen and stored at −80° C. To continue nuclear extract preparation, frozen nuclear extracts were thawed on ice and ammonium sulfate was added slowly to nuclear extracts to a final concentration of 0.35 mg/ml while stirring. Precipitated nuclear proteins were centrifuged in Sorval SS34 rotor at 16 k rpm for 30 min. The pellets were resuspended in Nuclear Extract Buffer (25 mM Hepes/KOH pH 7.6, 40 mM KCl, 0.1 mM EDTA, 10% glycerol, 5 mM β-mercaptoethanol) with 1 mM PMSF, 5 μg/ml antipain, 5 μg/ml leupeptin and 5 μg/ml aprotinin and dialyzed for 6 hours against NEB with 0.1 mM PMSF. The dialyzed nuclear extracts were aliquoted and stored at −80° C. until use. Gel Shift Assays

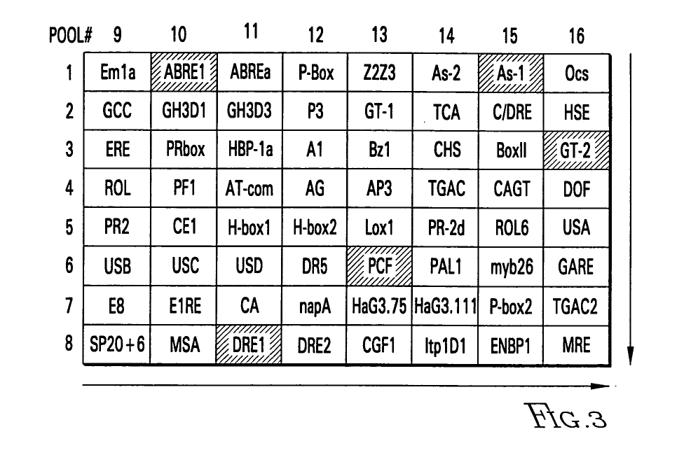

For DNA-binding reactions, about 1-2 μg aliquots of the nuclear extracts were incubated with the labeled DNA probes (10 ng) in presence of 1 μg poly(dl-dC). The binding reactions were incubated on ice for 5-20 minutes and run on 4% polyacrylamide-0.5 x TBE gel at room temperature for 2 hours. Each of the 16 lanes of the gel corresponded to one pool of oligonucleotides as indicated in FIG. 2. The gel was then dried and exposed to Kodak film. Gel shift results indicated that some oligo probes were bound very strongly by factors in maize nuclear extracts, as evidenced by their reduced mobility in the gel. Cross-reference to these strong binding activities from the two dimensional 8×8 pooling register (FIG. 2) indicated that these strong binding activities were contributed by the promoter elements PCNA IIA, GT-2, ABRE1, As-1, and DRE1, as indicated in FIG. 3.

### EXAMPLE 2

### Multimerization of Promoter Elements

Because promoter elements PCNA IIA, GT-2, ABRE1, As-1, and DRE1 were bound strongly by factors in maize nuclear extracts, the conclusion was that the transcription factors interacting with the elements are expressed abundantly in maize. Accordingly, the five promoter elements were selected to be synthetically combined into highly active synthetic promoters.

To synthesize the promoter element multimers, the oligos described above (top and bottom strands having spacer sequences) for the five promoter elements were phosphorylated by T4 DNA kinase (1 μg DNA in 10 μl reaction). Then these five pairs of oligos were annealed in separate reactions as described above. Five annealed oligo pairs were combined and ligated randomly into different promoter element multimer sequences in one reaction. Average size of ligated products was ˜200 bps. DNA from the ligation reaction was gel purified to remove small DNA fragments (˜100 bps and below) and unligated molecules. The ends of the purified DNA fragments were repaired by Klenow enzyme and cloned into expression vectors.

### EXAMPLE 3

### Cloning and Transient Assays of Synthetic Promoter Elements

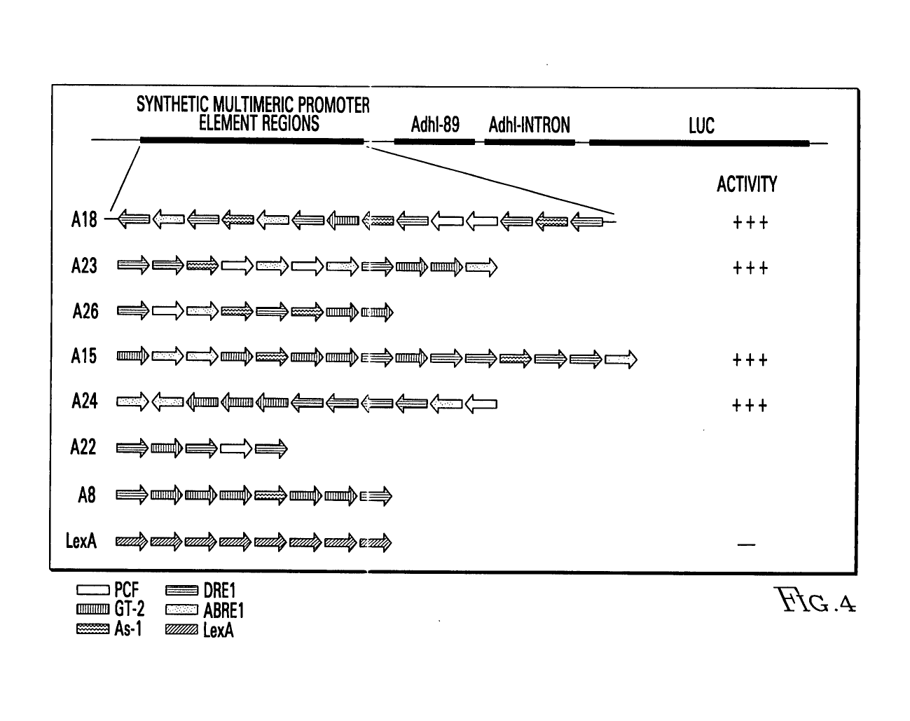

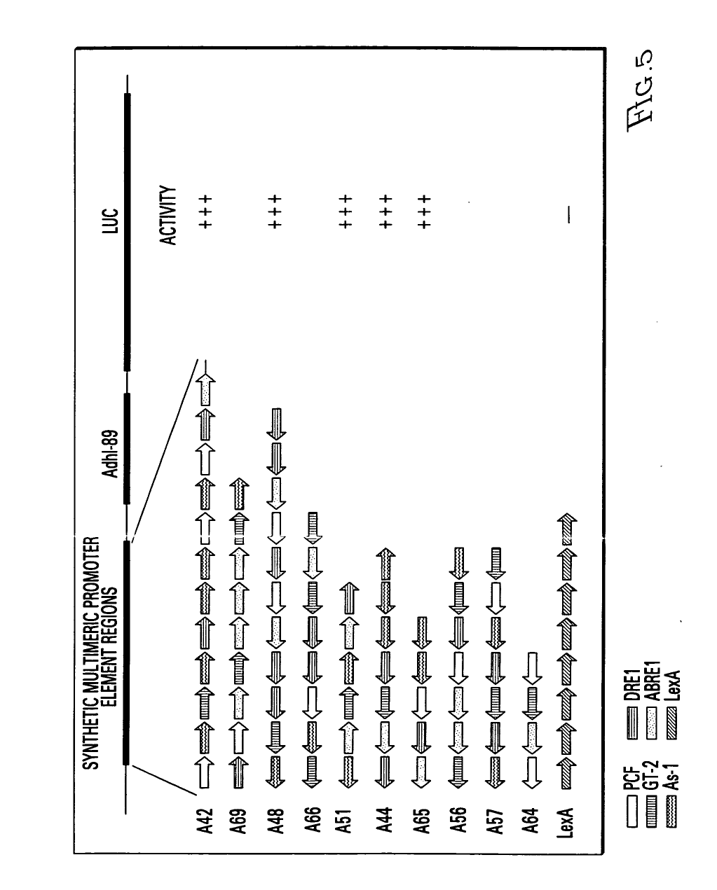

To clone the synthetic promoters into expression vectors, Adhl intron-plus plasmid P1 (LexA::Adhl-89-minimal::Adh intron::LUC::Pinll), and Adhl intron-minus plasmid P2 (LexA::Adhl-89-minimal::LUC::Pinll) were digested with restriction enzymes to remove the LexA promoter element sequences. The cleaved sites were filled by Klenow enzyme, and the resulting backbone vectors were gel purified. The synthetic promoters were ligated into these backbone expression vectors in separate reactions. About 20 positive clones for each construct were sequenced. Each ligated sequence was compared to the original promoter element sequences using either software Sequencher (GeneCodes, Ann Arbor, Mich.) or GAP (Genetics Computer Group (GCG), 575 Science Drive, Madison, Wis., USA). Based on the sequence information, seven constructs derived from Adhl intron-plus plasmids (FIG. 4) and ten constructs derived from Adhl intron-minus plasmids were chosen for transient expression analysis (FIG. 5).

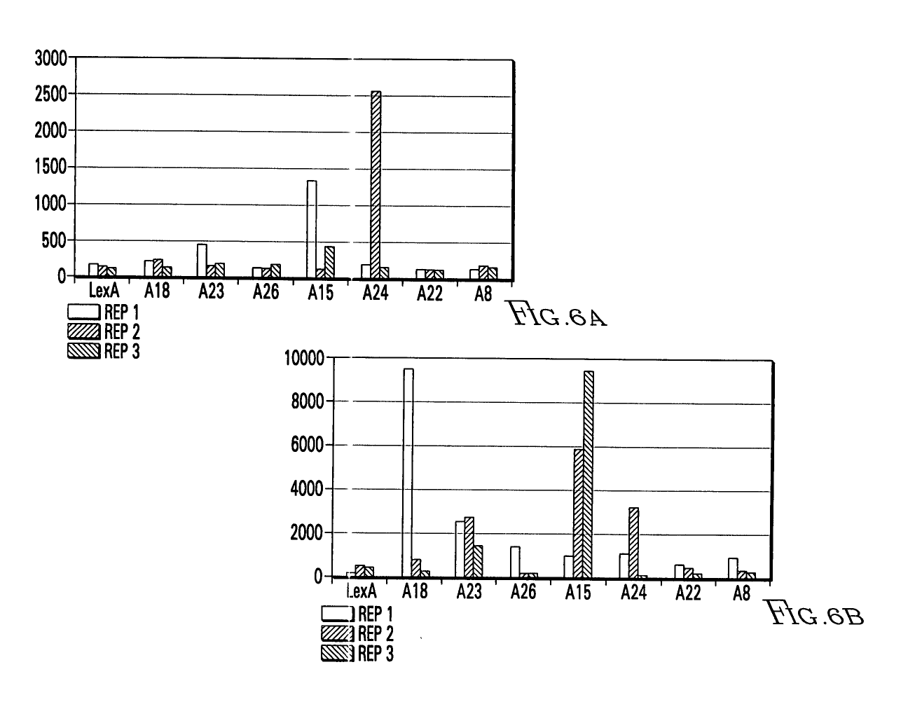

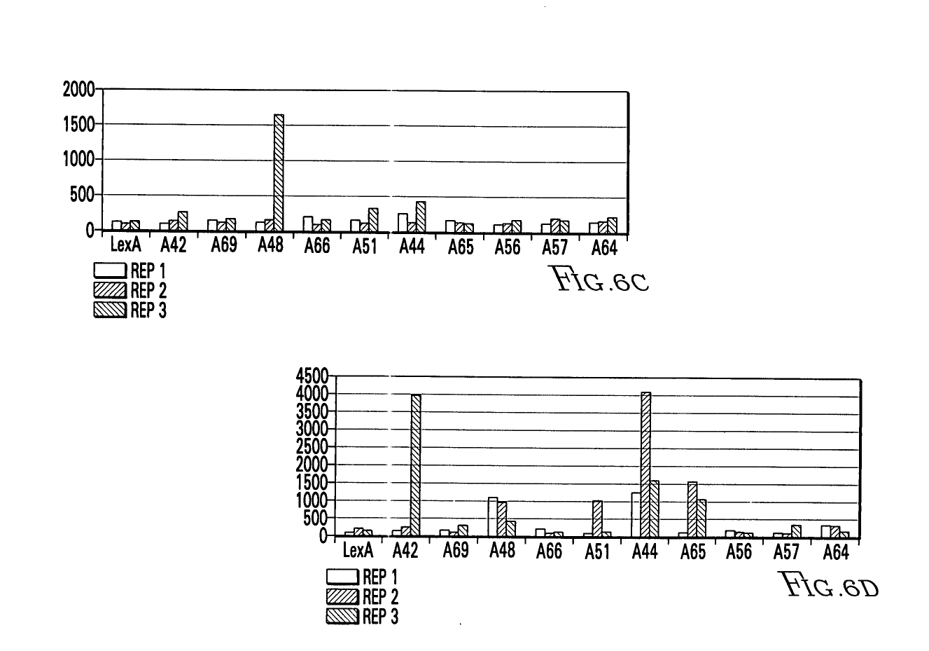

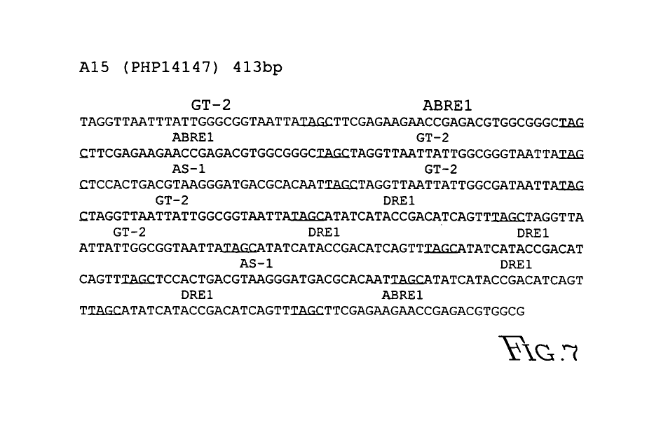

Three-day-old seedlings of maize were bombarded with 3 μg of the experimental plasmids comprising SMPER::Adhl-89-minimal::Adh intron::LUC::Pinll or those comprising SMPER::Adhl-89-minimal::LUC::Pinll. (See Tomes, D. et al., IN: Plant Cell, Tissue and Organ Culture: Fundamental Methods, Eds. O. L. Gamborg and G. C. Phillips, Chapter 8, pgs. 197-213 (1995), for general description of bombardment process.) Following 20 hours of incubation in the dark, crude protein extracts were prepared from roots and shoots. 20 μl tissue extracts were used for luciferase activity assays. For measurement of promoter activity, luciferase activity was used directly for each construct (FIG. 6). The negative controls (plasmids P1 and P2) and their derivatives without the LexA sequence showed very low activity. The transient assays indicated that synthetic promoters comprising certain SMPERs can promote gene expression in maize. Therefore, only unique combinations of promoter elements generate functional promoters. Some synthetic promoters comprising particular SMPERs (A 15, A18, A23, A24, A42, A44, A48, and A51 (FIG. 6; sequences in FIGS. 7-14 (SEQ ID NO: 65-72)) exhibited enhanced gene expression.

### EXAMPLE 4

### Transformation and Regeneration of Transgenic Maize

Biolistics:

The inventive polynucleotides contained within a vector are transformed into embryogenic maize callus by particle bombardment, generally as described by Tomes, D. et al., IN: Plant Cell, Tissue and Organ Culture: Fundamental Methods, Eds. O. L. Gamborg and G. C. Phillips, Chapter 8, pgs. 197-213 (1995) and as briefly outlined below. Transgenic maize plants are produced by bombardment of embryogenically responsive immature embryos with tungsten particles associated with DNA plasmids. The plasmids comprise a selectable marker gene and a structural gene of interest.

Preparation of Particles:

Fifteen mg of tungsten particles (General Electric), 0.5 to 1.8μ, preferably 1 to 1.8μ, and most preferably 1μ, are added to 2 ml of concentrated nitric acid. This suspension was sonicated at 0° C. for 20 minutes (Branson Sonifier Model 450, 40% output, constant duty cycle). Tungsten particles are pelleted by centrifugation at 10000 rpm (Biofuge) for one minute, and the supernatant is removed. Two milliliters of sterile distilled water are added to the pellet, and brief sonication is used to resuspend the particles. The suspension is pelleted, one milliliter of absolute ethanol is added to the pellet, and brief sonication is used to resuspend the particles. Rinsing, pelleting, and resuspending of the particles is performed two more times with sterile distilled water, and finally the particles are resuspended in two milliliters of sterile distilled water. The particles are subdivided into 250-ml aliquots and stored frozen.

Preparation of Particle-Plasmid DNA Association:

The stock of tungsten particles are sonicated briefly in a water bath sonicator (Branson Sonifier Model 450, 20% output, constant duty cycle) and 50 ml is transferred to a microfuge tube. All the vectors were cis: that is the selectable marker and the gene of interest were on the same plasmid. These vectors were then transformed either singly or in combination.

Plasmid DNA was added to the particles for a final DNA amount of 0.1 to 10 μg in 10 μL total volume, and briefly sonicated. Preferably, 10 μg (1 μg/μL in TE buffer) total DNA is used to mix DNA and particles for bombardment. Fifty microliters (50 μL) of sterile aqueous 2.5 M CaCl₂ are added, and the mixture is briefly sonicated and vortexed. Twenty microliters (20 μL) of sterile aqueous 0.1 M spermidine are added and the mixture is briefly sonicated and vortexed. The mixture is incubated at room temperature for 20 minutes with intermittent brief sonication. The particle suspension is centrifuged, and the supematant is removed. Two hundred fifty microliters (250 μL) of absolute ethanol are added to the pellet, followed by brief sonication. The suspension is pelleted, the supematant is removed, and 60 ml of absolute ethanol are added. The suspension is sonicated briefly before loading the particle-DNA agglomeration onto macrocarriers.

Preparation of Tissue:

Immature embryos of maize variety High Type II are the target for particle bombardment-mediated transformation. This genotype is the F₁ of two purebred genetic lines, parents A and B, derived from the cross of two know maize inbreds, A188 and B73. Both parents are selected for high competence of somatic embryogenesis, according to Armstrong et al., Maize Genetics Coop. News 65:92 (1991).

Ears from F₁ plants are selfed or sibbed, and embryos are aseptically dissected from developing caryopses when the scutellum first became opaque. This stage occurs about 9-13 days post-pollination, and most generally about 10 days post-pollination, depending on growth conditions. The embryos are about 0.75 to 1.5 millimeters long. Ears are surface sterilized with 20-50% Clorox for 30 minutes, followed by three rinses with sterile distilled water.

Immature embryos are cultured with the scutellum oriented upward, on embryogenic induction medium comprised of N6 basal salts, Eriksson vitamins, 0.5 mg/l thiamine HCl, 30 gm/l sucrose, 2.88 gm/l L-proline, 1 mg/l 2,4-dichlorophenoxyacetic acid, 2 gm/l Gelrite, and 8.5 mg/l AgNO₃. Chu et al., Sci. Sin. 18:659 (1975); Eriksson, Physiol. Plant 18:976 (1965). The medium is sterilized by autoclaving at 121° C. for 15 minutes and dispensed into 100×25 mm Petri dishes. AgNO₃ is filter-sterilized and added to the medium after autoclaving. The tissues are cultured in complete darkness at 28° C. After about 3 to 7 days, most usually about 4 days, the scutellum of the embryo swells to about double its original size and the protuberances at the coleorhizal surface of the scutellum indicate the inception of embryogenic tissue. Up to 100% of the embryos display this response, but most commonly, the embryogenic response frequency is about 80%.

When the embryogenic response is observed, the embryos are transferred to a medium comprised of induction medium modified to contain 120 gm/l sucrose. The embryos are oriented with the coleorhizal pole, the embryogenically responsive tissue, upwards from the culture medium. Ten embryos per Petri dish are located in the center of a Petri dish in an area about 2 cm in diameter. The embryos are maintained on this medium for 3-16 hours, preferably 4 hours, in complete darkness at 28° C. just prior to bombardment with particles associated with plasmid DNAs containing the selectable marker gene and structural gene or genes of interest.

To effect particle bombardment of embryos, the particle-DNA agglomerates are accelerated using a DuPont PDS-1000 particle acceleration device. The particle-DNA agglomeration is briefly sonicated and 10 ml were deposited on macrocarriers and the ethanol is allowed to evaporate. The macrocarrier is accelerated onto a stainless-steel stopping screen by the rupture of a polymer diaphragm (rupture disk). Rupture is effected by pressurized helium. The velocity of particle-DNA acceleration is determined based on the rupture disk breaking pressure. Rupture disk pressures of 200 to 1800 psi are used, with 650 to 1100 psi being preferred, and about 900 psi being most highly preferred. Multiple disks are used to effect a range of rupture pressures.

The shelf containing the plate with embryos is placed 5.1 cm below the bottom of the macrocarrier platform (shelf #3). To effect particle bombardment of cultured immature embryos, a rupture disk and a macrocarrier with dried particle-DNA agglomerates are installed in the device. The He pressure delivered to the device is adjusted to 200 psi above the rupture disk breaking pressure. A Petri dish with the target embryos is placed into the vacuum chamber and located in the projected path of accelerated particles. A vacuum is created in the chamber, preferably about 28 in Hg. After operation of the device, the vacuum is released and the Petri dish is removed.

Bombarded embryos remain on the osmotically-adjusted medium during bombardment, and 1 to 4 days subsequently. The embryos are transferred to selection medium comprised of N6 basal salts, Eriksson vitamins, 0.5 mg/1 thiamine HCl, 30 gm/l sucrose, 1 mg/l 2,4-dichlorophenoxyacetic acid, 2 gm/l Gelrite, 0.85 mg/l Ag NO₃ and 3 mg/l bialaphos (Herbiace, Meiji). Bialaphos is added filter-sterilized. The embryos are subcultured to fresh selection medium at 10 to 14 day intervals. After about 7 weeks, embryogenic tissue, putatively transformed for both the selectable marker gene and a structural gene or genes of interest, proliferates from about 7% of the bombarded embryos. Putative transgenic tissue is rescued, and that tissue derived from individual embryos is considered to be an event and is propagated independently on selection medium. Two cycles of clonal propagation are achieved by visual selection for the smallest contiguous fragments of organized embryogenic tissue.

A sample of tissue from each event is processed to recover DNA. The DNA is restricted with a restriction endonuclease and probed with primer sequences designed to amplify DNA sequences overlapping at least a portion of a synthetic multimeric promoter element region. Embryogenic tissue with amplifiable sequence is advanced to plant regeneration.

For regeneration of transgenic plants, embryogenic tissue is subcultured to a medium comprising MS salts and vitamins (Murashige & Skoog, Physiol. Plant 15: 473 (1962)), 100 mg/l myo-inositol, 60 gm/l sucrose, 3 gm/l Gelrite, 0.5 mg/l zeatin, 1 mg/l indole-3-acetic acid, 26.4 ng/l cis-trans-abscissic acid, and 3 mg/l bialaphos in 100×25 mm Petri dishes, and is incubated in darkness at 28° C. until the development of well-formed, matured somatic embryos can be seen. This requires about 14 days. Well-formed somatic embryos are opaque and cream-colored, and are comprised of an identifiable scutellum and coleoptile. The embryos are individually subcultured to a germination medium comprising MS salts and vitamins, 100 mg/l myo-inositol, 40 gm/l sucrose and 1.5 gm/l Gelrite in 100×25 mm Petri dishes and incubated under a 16 hour light:8 hour dark photoperiod and 40 meinsteinsm⁻² sec⁻¹ from cool-white fluorescent tubes. After about 7 days, the somatic embryos have germinated and have produced a well-defined shoot and root. The individual plants are subcultured to germination medium in 125×25 mm glass tubes to allow further plant development. The plants are maintained under a 16 hour light:8 hour dark photoperiod and 40 meinsteinsm⁻² sec⁻¹ from cool-white fluorescent tubes. After about 7 days, the plants are well-established and are transplanted to horticultural soil, hardened off, and potted into commercial greenhouse soil mixture and grown to sexual maturity in a greenhouse. An elite inbred line is used as a male to pollinate regenerated transgenic plants.

Agrobacterium-mediated Transformation:

As a preferred alternative to particle bombardment, plants are transformed using Agrobacterium-mediated transformation. To construct transgenic vectors for this transformation, the synthetic promoters contained in the transient assay vectors (Example 3) were transferred to transgenic vectors by appropriate restriction digestion and ligation. The promoter fragments isolated from Adhl intron-plus transient vector P1 derivatives and from Adhl intron-minus transient vector P2 derivatives were ligated into the backbone of the transgenic vector P3 (GUS::Pinll/2×CaMV35S::O′::Adhl intron::BAR::Pinll) upstream of the GUS reporter sequence. The backbone of vector P3 was prepared by digestion of vector P3 to remove ubiquitin (UBI) promoter, 5′ UTR, and UBI intron.

These resulting intermediate transgenic vectors were introduced into Agrobacterium tumefaciens LBA4404 by triparental matings to generate ‘super-binary’ vectors. Agrobaterium tumefaciens LBA4404 harboring the super-binary vector is used to transform maize.

For Agrobacterium-mediated transformation, the method of Zhao is employed (PCT patent publication WO98/32326, the contents of which are hereby incorporated by reference). Briefly, immature embryos are isolated from maize and the embryos contacted with a suspension of Agrobacterium (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step) and preferably calli grown on selective medium are cultured on solid medium to regenerate the plants. Regenerated plants are monitored and scored for the activity of the gene of interest.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. An isolated plant promoter comprising at least one synthetic multimeric promoter element region that is capable of driving transcription in a plant cell, wherein said promoter comprises a polynucleotide selected from the group consisting of:
(a) a nucleotide sequence of not greater than 2000 nucleotides comprising promoter elements GT-2 comprising SEQ ID NO.:24, ABRE1 comprising SEQ ID NO.:2, ABRE1 comprising SEQ ID NO.:2, GT-2 comprising SEQ ID NO.:24, As-1 comprising SEQ ID NO.:7, GT-2 comprising SEQ ID NO.:24, GT-2 comprising SEQ ID NO.:24, DRE1 comprising SEQ ID NO.:59, GT-2 comprising SEQ ID NO.:24, DRE1 comprising SEQ ID NO.:59, DRE1 comprising SEQ ID NO.:59, As-1 comprising SEQ ID NO.:7, DRE1 comprising SEQ ID NO.:59, DRE1 comprising SEQ ID NO.:59, and ABRE1 comprising SEQ ID NO.:2, sequentially;
(b) a nucleotide sequence comprising SEQ ID NO.:65;
(c) a nucleotide sequence of not less than 50 nucleotides that hybridizes under stringent conditions to a nucleotide sequence of (a) or (b), wherein said stringent conditions are hybridization in 50% formamide,1 M NaCl, 1% SDS at 37° C., and a wash in 0.1×SSC at 60-65° C.; and
(d) a polynucleotide which has at least about 90% sequence identity as determined by the GAP algorithm under default parameters across the full length of a sequence of (a) or to the promoter elements of (b).

2. A chimeric gene comprising the promoter of claim 1 operably linked to a coding sequence.

3. An expression cassette comprising the chimeric gene of claim 2.

4. A transformation vector comprising the expression cassette of claim 3.

5. A plant stably transformed with the transformation vector of claim 4.

6. A plant, or its parts, having stably incorporated into its genome a DNA construct comprising a plant promoter of claim 1 operably linked to a coding sequence.

7. A plant, or its parts, having stably incorporated into its genome a DNA construct comprising a plant promoter operably linked to a coding sequence, said plant promoter comprising at least one synthetic multimeric promoter element region that is capable of driving transcription in a plant cell, wherein said promoter comprises a polynucleotide selected from the group consisting of:
(a) a nucleotide sequence of not greater than 2000 nucleotides comprising promoter elements GT-2 comprising SEQ ID NO.:24, ABRE1 comprising SEQ ID NO.:2, ABRE1 comprising SEQ ID NO.:2, GT-2 comprising SEQ ID NO.:24, As-1 comprising SEQ ID NO.:7, GT-2 comprising SEQ ID NO.:24, GT-2 comprising SEQ ID NO.:24, DRE1 comprising SEQ ID NO.:59, GT-2 comprising SEQ ID NO.:24, DRE1 comprising SEQ ID NO.:59, DRE1 comprising SEQ ID NO.:59, As-1 comprising SEQ ID NO.:7, DRE1 comprising SEQ ID NO.:59, DRE1 comprising SEQ ID NO.:59, and ABRE1 comprising SEQ ID NO.:2, sequentially;
(b) a nucleotide sequence comprising SEQ ID NO.:65;
(c) a nucleotide sequence of not less than 50 nucleotides that hybridizes under stringent conditions to a nucleotide sequence of (a) or (b), wherein said stringent conditions are hybridization in 50% formamide, 1 M NaCL, 1% SDS at 37° C., and a wash in 0.1×SSC at 60-65° C.; and
(d) a polynucleotide which has at least about 90% sequence identity as determined by the GAP algorithm under default parameters across the full length of a sequence of (a) or to the promoter elements of (b).

8. The plant of claim 7, wherein said plant is a dicot.

9. The plant of claim 7, wherein said plant is a monocot. 10. The plant of claim 9, wherein said monocot is maize.

11. A plant cell having stably incorporated into its genome a DNA construct comprising a plant promoter operably linked to a coding sequence, said plant promoter comprising at least one synthetic multimeric promoter element region that is capable of driving transcription in a plant cell, wherein said promoter comprises a polynucleotide selected from the group consisting of:
(a) a nucleotide sequence of not greater than 2000 nucleotides comprising promoter elements GT-2 comprising SEQ ID NO.:24, ABRE1 comprising SEQ ID NO.:2, ABRE1 comprising SEQ ID NO.:2, GT-2 comprising SEQ ID NO.:24, As-1 comprising SEQ ID NO.:7, GT-2 comprising SEQ ID NO.:24, GT-2 comprising SEQ ID NO.:24, DRE1 comprising SEQ ID NO.:59, GT-2 comprising SEQ ID NO.:24, DRE1 comprising SEQ ID NO.:59, DRE1 comprising SEQ ID NO.:59, As-1 comprising SEQ ID NO.:7, DRE1 comprising SEQ ID NO.:59, DRE1 comprising SEQ ID NO.:59, and ABRE1 comprising SEQ ID NO.:2, sequentially;
(b) a nucleotide sequence comprising SEQ ID NO.:65;
(c) a nucleotide sequence of not less than 50 nucleotides that hybridizes under stringent conditions to the nucleotide sequence of (a) or (b), wherein said stringent conditions are hybridization in 50% formamide, 1 M NaCl,1% SDS at 37° C., and a wash in 0.1×SSC at 60-65° C.; and,
(d) a polynucleotide which has at least about 90% sequence identity as determined by the GAP algorithm under default parameters across the full length of a sequence of (a) or to the promoter elements of (b).

12. The plant cell of claim 11, wherein said plant cell is from a dicotyledonous plant.

13. The plant cell of claim 11, wherein said plant cell is from a monocotyledonous plant.

14. The plant cell of claim 13, wherein said monocotyledonous plant is a maize plant.

15. A method for constitutively expressing a heterologous nucleotide sequence in a plant, said method comprising:
(a) transforming a plant cell with a transformation vector comprising an expression cassette, said expression cassette comprising a plant promoter of claim 2 operably linked to a coding sequence; and
(b) regenerating a stably transformed plant from said transformed cell, said plant having stably incorporated into its genome said expression cassette.
